(19) United States
(12) Patent Application Publication
(10) Pub. No.: US 2013/0196406 A1
(43) Pub. Date: Aug. 1, 2013

(54) NOVEL PROTEIN-POLYMER NANOCAPSULES

(71) Applicant: University of Connecticut, Farmington, CT (US)

(72) Inventors: Challa V. Kumar, Ashford, CT (US); Rajeswari M. Cozy, Ellington, CT (US); Vamsi K. Mudhivarthi, Storrs, CT (US)

(73) Assignee: University of Connecticut, Farmington, CT (US)

(21) Appl. No.: 13/748,973

(22) Filed: Jan. 24, 2013

Related U.S. Application Data
(60) Provisional application No. 61/591,617, filed on Jan. 27, 2012.

(51) Int. Cl. C07K 17/08; C12N 9/96

(52) U.S. Cl. CPC C07K 17/08; C12N 9/96
USPC 435/188; 525/54.1; 428/402

(57) ABSTRACT

Disclosed are protein nanocapsules, and in particular, nanocapsules that have high thermal stability and very high resistance to thermal inactivation when subjected to steam sterilization.

### CROSS-REFERENCE

This application claims priority to U.S. Provisional Patent Application Ser. No. 61/591,617 filed Jan. 27, 2012, incorporated by reference herein in its entirety.

### STATEMENT REGARDING FEDERALLY SPONSORED RESEARCH OR DEVELOPMENT

This work was funded with government support under BMAT-1005609 awarded by National Science Foundation. The U.S. government has certain rights in the invention.

### BACKGROUND OF THE INVENTION

Protein-polymer conjugates are promising agents for improving the stability and properties of therapeutic proteins. Partly, this is because of the preservation of the first few layers of hydration surrounding the protein, improved solubility in water and physically shielding the protein from proteolytic enzymes. A number of approaches are being developed for the attachment of polymers to proteins, and majority of these rely on site-specific attachment. Such polymer-protein conjugates provide avenues to introduce biological function into synthetic polymer-protein nanoparticles and the role of polyethylene glycol spacers have been examined. Attachment of biodegradable polymers to proteins provided control over polymer degradation and protein release. Alternatively, proteins are being attached to hydrophobic polymers with retention of significant activity and storage stability. Shielding of the protein from the external microenvironment could enhance the intrinsic stability of the protein.

### SUMMARY OF THE INVENTION

One aspect of the disclosure provides a nanocapsule comprising a protein moiety covalently attached to a polymer, wherein the polymer encapsulates the protein in a nanoshell.

Another aspect of the disclosure provides processes for preparation of the nanocapsules of the disclosure.

One aspect provides use of nanocapsules of the disclosure in biomaterials, bioreactors or biomedical applications.

### BRIEF DESCRIPTION OF DRAWINGS

FIG. 1 provides verification of conjugation by agarose gel electrophoresis of Hb (lane 1), Hb-PAA-1k (lanes 2, 3 and 4), and Hb-PAA-10k (lanes 5, 6 and 7). FIG. 1A provides verification of conjugation by agarose gel electrophoresis of carbonic anhydrase (CA, lane 1), CA-PAA-1k (lane 2, pH 6), CA-PAA-10k (lane 3, pH 6), glucose oxidase (GO, lane 5), GO-PAA-1k (lane 6, pH 6), and GO-PAA-10k (lane 7, pH 6).

FIG. 2 shows TEM images of Hb-PAA-1k and Hb-PAA-10k samples synthesized at pH 6, 7 or 8 after ruthenium oxide staining: a) Hb-PAA-1k-6, b) Hb-PAA-1k-7, c) Hb-PAA-1k-8, d) Hb-PAA-10k-6, e) Hb-PAA-10k-7 and f) Hb-PAA-10k-8.

FIG. 3 provides a) far UV CD of Hb, Hb-PAA-1k conjugates synthesized at pH 6 (red), 7 (green), and 8 (blue) when compared to that of untreated Hb (black); and b) Soret CD of the Hb-PAA-1k samples with the same color code as A. All spectra were normalized to obtain molar rotation per unit path length.

FIG. 4 provides representative kinetic traces showing the peroxidase-like activities of Hb-PAA-1k and Hb-PAA-10k nanocapsules synthesized at pH 6 and 7, respectively. Specific activities of Hb, physical mixtures of Hb/PAA and samples without hydrogen peroxide (under the same conditions of buffer, pH and temperature) are compared with those of the nanocapsules. All experiments were done in the presence of 1 μM protein, 2.5 mM guaiacol and 1 mM H₂O₂ in 20 mM Na₂HPO₄ buffer, pH 7.2.

FIG. 5 provides Lineweaver-Burk plots for the peroxidase-like activity of Hb-PAA conjugates. A) Peroxidase-like activity for the 1 μM of Hb-PAA synthesized at pH 6.0. B) Peroxidase-like activity for 1 μM Hb-PAA synthesized at pH 7.0. All experiments were done in the presence of 1 μM Hb, 2.5 mM guaiacol and 1 mM H₂O₂.

FIG. 6 shows a) melt-curves of Hb, Hb/PAA and Hb-PAA-1k-7 recorded by monitoring heme absorption at 407 nm as a function of temperature; and b) The DSC thermograms (molar heat capacity plots) of Hb (thick solid line), Hb/PAA physical mixture (dotted line) and Hb-PAA-1k-7 (green curve) as a function of temperature.

FIG. 7 shows far UV CD of Hb, Hb-PAA-1k conjugates heated to 90° C. and cooled to room temperature for 15 min, indicated in black and red dashed lines respectively.

FIG. 8 illustrates effect of steam sterilization on the peroxidase-like activities of Hb (green), Hb/PAA physical mixture (blue) and Hb-PAA nanocapsules synthesized at pH 6, 7 and 8 (red), (a) 1k, and (b) 10k samples. All activities were measured with 1 μM Hb, 2.5 mM guaiacol and 1 mM H₂O₂ in 20 mM Na₂HPO₄ buffer, pH 7.2, room temperature.

### DETAILED DESCRIPTION OF THE INVENTION

In a first aspect, the present disclosure provides nanocapsules comprising a protein moiety covalently attached to a polymer, wherein the polymer encapsulates the protein in a nanoshell.

The nanocapsules may comprise discrete cavities in its interior, or may be a porous network. In various embodiments, the nanocapsule has a mean diameter in one or more of the ranges between: about 1 nm to about 300 nm; about 10 nm to about 200 nm; about 10 nm to about 100 nm; and about 50 nm to about 100 nm; about 100 nm to about 200 nm. It is to be understood that the term “mean diameter” is not meant to imply any sort of specific symmetry (e.g., spherical, ellipsoidal, etc.) of a nanocapsule. Rather, the nanocapsule could be highly irregular and asymmetric.

In one embodiment, the disclosure provides nanocapsules as described above wherein the nanoshell is a thin film of polymer. The thin layer of polymer is thick enough to protect the protein moiety while allowing for the ingress/egress of the substrate molecules or the corresponding ligands to interact with the protein moiety. Thus, in several embodiments, the thin film of polymer has thickness in one or more ranges between: about 1 nm to about 100 nm; about 10 nm to about 50 nm; about 10 nm to about 100 nm; about 50 nm to about 100 nm; about 80 nm to about 100 nm. The thickness may or may not be uniform.

The nanocapsule can comprise hydrophilic (ionized, ionizable, or polar non-charged) and hydrophobic regions.

Methods of encapsulating the protein moiety are well known in the art. Encapsulating may be done through attaching (i.e., crosslinking) the polymer groups to the protein moiety via covalent bonds or by associating the polymer groups to the protein moiety via non-covalent interactions.

Thus, in one embodiment, the disclosure provides nanocapsules as described above wherein the polymer is covalently bound (i.e., attached or crosslinked) to the protein moiety such as that the polymer encapsulates the protein moiety. In another embodiment, the protein moiety is covalently attached to the polymer at one or more sites on the protein and at one or more sites on the polymer, such as that the polymer encapsulates the protein moiety. In yet another embodiment, the protein moiety is covalently attached to the polymer at two or more sites on the protein and at two or more sites on the polymer, such as that the polymer encapsulates the protein moiety. In one embodiment, the disclosure provides nanocapsules wherein the attachment of the protein to the polymer is through a specific site on the protein, which can be located anywhere on the protein (i.e., random) or can be specifically located on the protein (i.e., selective). Thus in one embodiment, the disclosure provides nanocapsules wherein the attachment of the protein to the polymer is random. In another embodiment, the disclosure provides nanocapsules wherein the attachment of the protein to the polymer is selective.

Crosslinking may occur on a specific location within the nanocapsule, or across the entire nanocapsule. Examples of crosslinking reactions include, but are not limited to e.g., esterification, amidation, addition, or condensation reactions. Crosslinking can be induced by light, temperature, stoichiometric reagents, or the presence of a catalyst, or crosslinking can be spontaneous. Crosslinking reaction can be induced in the presence of a catalyst or a coupling reagent. Such coupling reagents include, but are not limited to, carbodiimide compounds, e.g., N,N′-dicyclohexylcarbodiimide, N,N′-diisopropylcarbodiimide, and 1-ethyl-3-(3-dimethylaminopropyl)carbodiimide (aka N-(3-dimethylaminopropyl)-N′-ethylcarbodiimide hydrochloride or “EDC”). Cross-linking reaction can also be induced by the addition of an external crosslinker with or without the presence of a catalyst. Examples of external crosslinkers used to cross-link PAA, for example, include, but are not limited to, difunctional or polyfunctional alcohol (e.g. ethylene glycol, ethylenedioxy-bis(ethylamine), glycerol, polyethylene glycol), difunctional or polyfunctional amine (e.g, ethylene diamine, JEFFAMINE®), polyetheramines (Huntsman), poly(ethyleneimine)). Crosslinking reaction can be induced in the presence light (e.g., photochemical reaction). Light-induced crosslinking can be triggered by UV and visible light of various wavelengths, in air or under an inert environment, with or without photoinitiators. Examples of photoinitiators that activate in the UV wavelength region include, but are not limited to, phenyl bis(2,4,6-trimethylbenzoyl)-phosphine oxide (IRGACURE 819, Ciba Corporation), acetophenone, and benzophenones such as 2-hydroxy-2-methylpropiophenone. Examples of photoinitiators that activate in the visible wavelength region include, but are not limited to, benzil and benzoin compounds, and camphorquinone. The degree of crosslinking can be modified to control the transport of material from the interior of the nanocapsule to the environment of the nanocapsule. Crosslinking reaction can be induced in the presence of a protein, an enzyme, or other reagent. Crosslinking reaction can be spontaneous; i.e., where the crosslinking reaction is not induced by light or by temperature, or where the crosslinking reaction is in the absence of catalyst, external crosslinker, protein, or enzyme.

In certain embodiments, the binding of the polymer to the protein moiety can also be tuned or modified. This can be accomplished by modification of the surface chemistry of the polymer. Modification of the polymer can be performed by various methods, including conjugation, copolymerization, grafting and polymerization, or by exposure to free radicals. Modification can be designed before, during or after the preparation of nanocapsules. An example of polymer modification during the preparation of nanocapsules involves modification of poly(acrylic acid). Under appropriate conditions, poly(acrylic acid) that is exposed to UV will decarboxylate some of its acid groups, thereby increasing the hydrophobicity of the system. Similar treatment can be used with other types of polymers. Modification of the polymer can be observed using titration, spectroscopy or nuclear magnetic resonance (NMR) under suitable conditions. Polymer modification can also be observed using size exclusion or affinity chromatography. The hydrophobic and hydrophilic regions of the polymer nanoparticle can be observed using solvent effects.

In one embodiment, the disclosure provides nanocapsules as described above wherein the polymer is covalently bound to the protein moiety via an amino acid on the protein moiety. In another embodiment, the location of the amino acid on the protein is random (i.e., anywhere on the protein.) In yet another embodiment, the location of the amino acid on the protein is selective.

Thus, in one embodiment, the attachment of the protein to the polymer is through a lysine side chain of the protein moiety. In that embodiment, the polymer contains carboxylic acid groups (i.e., —COOH). In another embodiment, the attachment of the protein to the polymer is through a glutamate or aspartate side chain of the protein moiety. In that embodiment, the polymer contains amine groups (i.e., —NH₂).

In one embodiment, the disclosure provides nanocapsules as described above wherein the polymer is covalently bound to the protein moiety via a non-amino acid on the protein moiety. For example, the attachment of the protein to the polymer is through a prosthetic group, glycosylation site, phosphorylated site or chemically modified amino acid side chain.

In one embodiment, the protein moiety can also be physically or chemically associated with the polymer in a non-covalent fashion. Physical association is defined by non-covalent interactions such as charge-charge interactions, hydrophobic interactions, polymer-chain entanglement, affinity pair interactions, hydrogen bonding, van der Waals forces, or ionic interactions.

In one embodiment, the disclosure provides the nanocapsule as described above wherein the polymer is any suitable synthetic polymer or natural polymer that has acidic or basic functional groups.

In another embodiment, the disclosure provides the nanocapsules as described above wherein the polymer is a polyacid. Non-limiting examples of polyacids include polyacrylic acid, polyacrylic acid sodium salt, poly(acrylic acid-co-maleic acid), poly(methyl vinyl ether-alt-maleic acid), poly(acrylamide-co-acrylic acid), poly(lactic acid), poly(glycolic acid), or combinations thereof. In another embodiment, the nanocapsule of the disclosure as described above is wherein the polymer is a polyacrylic acid.

In one embodiment, the disclosure provides the nanocapsules as described above wherein the polymer has a molecular weight between about 500 and about 20,000. In another embodiment, the molecular weight is between about 5,000 and about 20,000. In one embodiment, the molecular weight is between about 5,000 and about 15,000; in another embodiment, the molecular weight is between about 5,000 and about 10,000. In another embodiment, the molecular weight is between about 10,000 and about 20,000. In other embodiment, the molecular weight is between about 15,000 and about 20,000. In another embodiment, the molecular weight is about 18,000.

In one embodiment, the disclosure provides the nanocapsules as described above wherein the protein includes one or more proteins, or a combination of two or more proteins, or a protein complex. In another embodiment, the protein is a single protein. In one embodiment, the protein is globular protein. Non-limiting examples include enzymes, hormones, membrane transport proteins, signal transduction proteins, immunoglobulins, alpha and beta globulins, globins, peptides and other proteins etc. In one embodiment, the disclosure provides the nanocapsules as described above wherein the protein is an enzyme. In another embodiment, the disclosure provides the nanocapsule as described above wherein the protein is a globin. In yet another embodiment, the disclosure provides the nanocapsule as described above wherein the protein is an antibody.

In one embodiment, the disclosure provides the nanocapsules as described above wherein the protein is met-hemoglobin, hemoglobin, glucose oxidase, carbonic anhydrase, or lipase.

In one embodiment, the disclosure encompasses the nanocapsules as described above where a thin, flexible, permeable, inert, polymeric nanoshell is constructed around the protein by conjugation with the polymer or mixtures of polymers. Such nanocapsules have (1) long shelf life of the protein, and (2) high thermal stability and resistance to denaturation when subjected to steam denaturation.

The formation of the nanocapsules of the disclosure can be determined using dynamic light scattering (DLS), atomic force microscopy (AFM) or transmission electron microscopy (TEM). In DLS, formation of the nanocapsules is demonstrated by an increase in average particle size relative to either the particle size of the protein or the particle size of the polymer encapsulant at the same concentration. In TEM or AFM the nanoparticles can be visualized directly.

### Therapeutics Applications

Improved protein stability is important for biomedical applications where sterilized materials are required. Thus, the disclosure provides use of the nanocapsules as described above for use in, for example, biomaterials, bioreactors or biomedical applications such as implants, drug delivery, surgical sutures, and coatings. In addition to the benefits of the nanocapsules for such use, the nanocapsules can be sterilized, for example by steam sterilization, and thus provide even further advantages.

The disclosure also provides methods of treating cancer the method comprising administering to a subject in need of such treatment an effective amount of one or more nanocapsules of the invention. Non-limiting examples of cancer include oral cancer, head, and neck cancer. Glucose oxidase can generate hydrogen peroxide, which can be lethal to cells, carbonic anhydrase can alter the pH in the cell due to dissolved CO₂ and upset the metabolism, and lipase is critical in the lipid metabolism. Thus, non-limiting examples of nanocapsules useful in treating cancer include glucose oxidase-PAA, hemoglobin-PAA, carbonic anhydrase-PAA, and lipase-PAA nanocapsules.

### Pharmaceutical Compositions

In another aspect, the present disclosure provides compositions comprising one or more of nanocapsules as described above and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

The nanocapsules of the disclosure can be formulated in a variety of ways. In some cases they can be dried into a solid by freeze drying, spray drying, tray drying, air drying, vacuum drying, or other drying methods. Once dried, they can be stored for some length of time and then re-suspended into a suitable solvent when they need to be used. In certain embodiments, the dried solid can be granulated, made into tablets, for handling.

The nanocapsules described herein may be administered singly, as mixtures of one or more nanocapsules or in mixture or combination with other agents useful for treating particular diseases and/or the symptoms associated with such diseases. The nanocapsules may also be administered in mixture or in combination with agents useful to treat disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The nanocapsules may be administered in the form of nanocapsules per se, or as pharmaceutical compositions comprising a nanocapsule.

The pharmaceutical compositions may, if desired, be presented in a pack or dispenser device which may contain one or more unit dosage forms containing the nanocapsules. The pack may, for example, comprise metal or plastic foil, such as a blister pack. The pack or dispenser device may be accompanied by instructions for administration.

### DEFINITIONS

The following terms and expressions used herein have the indicated meanings.

The term “encapsulate” or “encapsulation”, as used herein, refers to entrapment of a protein or another small molecule within the boundaries (confines) of a scaffold, which is a polymer layer in which the protein or another small molecule are embedded.

The term “nanocapsule” as used herein, refers to discrete polymer structure surrounding a core, which can be a protein or another small molecule. Such structure, can be symmetrical (e.g., spherical, ellipsoidal, etc.) or assymetric. Nanocapsules may or may not aggregate, but they are discrete entities.

The term “macrogel” or “nanogel” as used herein, refers to a polymer network that contains crosslinked polymer chains where the crosslinks are both between and within the polymer chains. Depending on the degree of crosslinking between the polymer chains, the polymer network is more or less entangled.

The term “nanoshell” as used herein, refers to a discrete polymer structure, which is not a polymer network, wherein there is no core.

The term “protein” or “protein moiety” as used herein, means a plurality of amino acid residues (generally greater than 10) joined together by peptide bonds, and has a molecular weight greater than 0.5 kDa, preferably greater than 5 kDa. This term is also intended to include fragments, analogues and derivatives of a protein wherein the fragment, analogue or derivative retains essentially the same biological activity or function as a reference protein. The protein may be a linear structure or a non-linear structure having a folded, for example tertiary or quaternary, conformation. The protein may have one or more prosthetic groups conjugated to it, for example the protein may be a glycoprotein, lipoprotein or chromoprotein. Preferably, the protein is a biologically active protein. For example, the protein may be selected from the group consisting of glycoproteins, serum albumins and other blood proteins, hormones, enzymes, receptors, antibodies, interleukins and interferons.

“Pharmaceutically acceptable” refers to those nanocapsules, compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

### EXAMPLES

The preparation of the nanocapsules of the disclosure is illustrated further by the following examples, which are not to be construed as limiting the disclosure in scope or spirit to the specific procedures and nanocapsules described in them.

### Example 1

### Synthesis of Hb-PAA Nanocapsules

Polyacrylic acid (PAA) (8000 Da) was purchased from sigma chemical Co (St. Louis, Mo.). The PAA stock solution was made by mixing 1.32 gm of 45% (w/w) PAA in 3 ml of 20 mM Na₂HPO₄ buffer, pH 7.2, and was further used for synthesis. Met-hemoglobin (Hb, bovine) was purchased from Sigma. Protein solutions were prepared in the respective buffers used for synthesis and the concentrations determined from the Soret absorbance of Hb using extinction co-efficient 303,956 M⁻¹ cm⁻¹ at 407 nm.

Covalent attachment of the carboxyl functions of PAA (MW 8000, 1 mmol or 10 mmol) to the lysine side chains of Hb was carried out by 1-ethyl-3-(3-dimethylaminopropyl)carbodiimide as the condensing agent (EDC, 100 mmol) at pH 6, 7 or 8, in 20 mM Na₂HPO₄ buffer. In particular, PAA 8 or 80 mg (1 or 10 mmol, 8000 MW) is dissolved in 1 ml of 20 mM Na₂HPO₄ buffer of pH 6, 7 or 8 buffers and was sonicated for 10 minutes before using it for the synthesis. Then 8 or 80 mg (1 or 10 mmol) PAA was taken into a vial and the volume was made to 900 μl with the respective buffer, followed by the addition of 100 mmol of EDC, while stirring. The resulting PAA, EDC mixture was stirred for 10 minutes and then 100 μl of 100 μgM Hb (10 μmol) was added and stirred for 12 h. Excess EDC, PAA and urea (byproduct) were dialyzed out 3 times with 1000 times the volume of sample using 15,000 D, dialysis membrane to obtain pure protein-polymer conjugate. The resulting conjugate is then centrifuged at 12,000 RPM and filtered using 0.22 μm filter, before use.

The product obtained with PAA (1000 mmol, given in terms of COOH groups, pH 7) reacted with Hb (10 gmol/ml) is designated as Hb-PAA-1k-7, while the reaction of 10000 mmol PAA with Hb (10 gmol/ml) at pH 7 resulted in Hb-PAA-10k-7. This nomenclature will be used throughout for unique identification of particular Hb-PAA nanocapsules. Under these reaction conditions, the entire reaction mixture remained soluble and there has been no precipitation at any time.

Covalent conjugation of carbon rich PAA polymer to Hb is expected to increase the C:N ratio, and from this ratio, one can assess the amount of polymer bound per protein molecule. For example, the C:N ratio of 6.34 was measured for the Hb-PAA-10k-8 which increased from 3.51 for the untreated Hb. Since the nanocapsules were extensively dialyzed to remove unreacted PAA, the increase in C:N ratio is indicative of the ratio of stoichiometry of Hb to PAA in the nanocapsule. This ratio indicated that on an average, nearly 7 PAA polymer chains were attached to every Hb, giving a Hb:PAA stoichiometry of 1:7. Thus, each protein molecule is surrounded by multiple polymer chains, and PAA formed an ultra-thin film of polymer around Hb.

### Example 2

### Agarose Gel Electrophoresis

Native agarose gels were prepared by dissolving agarose (0.5% w/v) (Sigma electrophoresis grade) in a hot solution of Tris acetate (40 mM, pH 6.5). The gel was run in a horizontal gel electrophoresis apparatus (Gibco model 200, Life Technologies Inc, MD) using Tris acetate (40 mM pH 7) as the running buffer. Samples were loaded into the gel with loading buffer (50% v/v glycerol and 0.01% m/m bromophenol blue) and electrophoresis carried out for 30 min at 100 mV at room temperature. The gel was stained overnight with 10% v/v acetic acid, 0.02% m/m Comassie blue and then de-stained with 10% v/v acetic acid for an additional night. The gel was photographed using a Molecular Imager Gel Doc XR System.

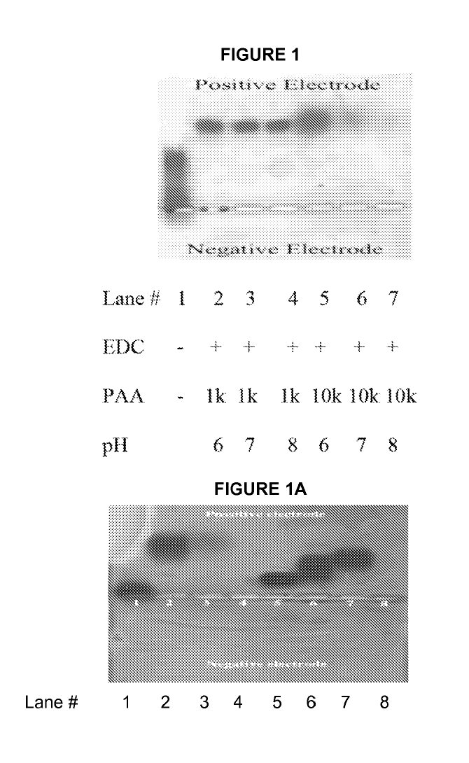

Covalent conjugation of Hb to PAA was verified by agarose gel electrophoresis where the attachment of anionic PAA is expected to increase the net negative charge and increase electrophoretic mobility. On the other hand, formation of macroscopic gels would retard the migration through the narrow pores of the gel. Hb moved as a streak (FIG. 1, lane 1) due to its aggregation, and Hb-PAA samples (FIG. 1, lanes 2-7) did not indicate any bands that correspond to Hb. The product bands, in fact, moved further toward the positive electrode when compared to Hb (FIG. 1, lane 1), and this is a direct evidence of increased negative charge on Hb-PAA conjugates due to the negatively charged carboxyl groups of PAA. The physical mixture of Hb/PAA did not indicate any influence of the polymer on Hb mobility. Four of the Hb-PAA samples indicated tight bands (FIG. 1, lanes 2-5), and these clearly indicated the formation of discrete protein-polymer adducts. Two of the samples (FIG. 1, lanes 6 and 7) had more diffuse bands due to higher concentrations of PAA used, but none of these lanes indicated the presence of macroscopically crosslinked gels, which would have produced streaks. Similar observations are made in the experiments with carbonic anhydrase (CA, CA-PAA-1k, CA-PAA-10k) and glucose oxidase (GO, GO-PAA-1k, GO-PAA-10k) (FIG. 1A).

Electrophoretic mobilities of Hb-PAA indicated a measure of their size. The average pore size of agarose gel used in FIG. 1 has been made of 0.5% gel (in water) is around 450 nm, and all the Hb-PAA conjugates moved readily through these pores. Therefore, the conjugates are smaller than 450 nm in diameter, there is no formation of macroscopically crosslinked gels, which would have indicated streaks of protein bands or precipitation in the wells. This conclusion is consistent with the observed high solubility of the conjugates in the reaction mixture and the low overlap concentration of low molecular weight of PAA (8,000 D). The gel confirms the clean ligation of Hb to PAA with particle size well below 450 nm. Formation of large macroscopic networks is unlikely at these low polymer and protein concentrations (1 mM and 10 mM). These samples were further analyzed for Hb and PAA contents to establish the composition of the nanocapsules.

### Example 3

### TEM and DLS Data

TEM was used to examine the morphology of the Hb-PAA conjugate, Hb and PAA mixture (Hb/PAA), Hb and PAA. The images were obtained with a Tecnai T12 instrument operating at an accelerating voltage of 120 kV. Solutions of Hb-PAA, Hb/PAA and PAA were diluted to 0.1 mg/ml based on PAA concentration. The Hb solution was diluted to 0.1 mg/ml Hb. A drop of each solution was deposited on a copper grid covered with Fomvar film. Excess solution was blotted away with a piece of filter paper to leave a thin layer of solution on the grid. The sample was left to dry in air, and then stained with ruthenium tetroxide for 30 min. Digital images were collected and presented.

The absorption spectra were recorded on HP 8453 spectrophotometer. The Hb solutions mixed in the respective buffers was determined from the Soret absorbance of Hb using extinction co-efficient 303,956 M⁻¹ cm⁻¹ at 407 nm. Further, the melting temperature of Hb, Hb/PAA and Hb-PAA were determined by measuring the transition points from the plots of Soret absorbance at 407 nm as a function of temperature.

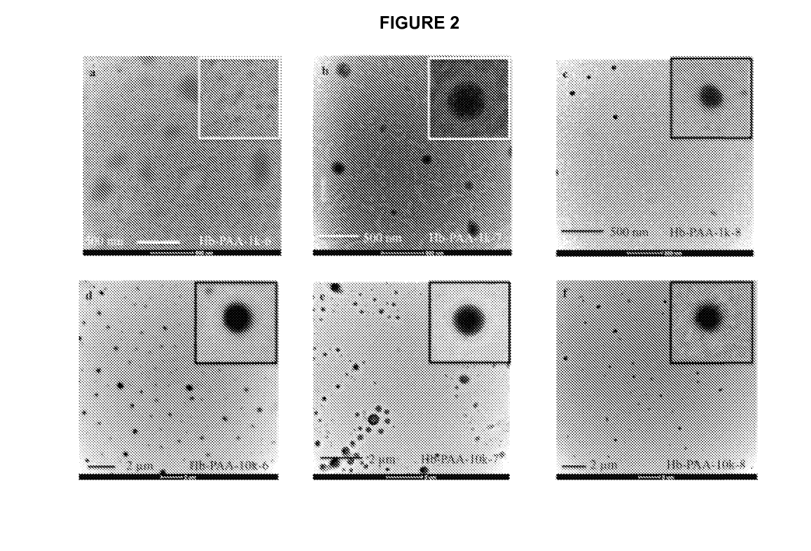

The Hb-PAA samples were directly visualized by TEM to determine their morphology, size, shape and phase separation characteristics. The micrographs indicated nearly spherical particles and there are no macrogels or highly crosslinked products (FIG. 2). In particular, Hb-PAA-1k synthesized at pH 7 and 8 (FIGS. 2b and 2c) are nearly spherical with a dark core and light corona with sizes in the range of 110-150 nm. The TEM micrographs of Hb-PAA-10k samples also showed nanoscopic species (FIG. 2, d-f), and indicated a significantly narrower size distribution of 80-100 nm. These are not macroscopic gels, and they indicated nanoscopic species with a spherical shape and narrow size distribution.

All the micrographs clearly display a light corona around the dark core of the particles. This observation suggests a core-shell model where the protein is segregated at the core of the particle, stained dark by ruthenium oxide which is surrounded by hydrophilic PAA outer shell which does not stain well. Phase separation of the highly water soluble polymer and moderately hydrophobic Hb nanodomains, where the protein nanoparticle is stabilized by the hydrophilic polymer shell, explains current observations. The TEM data are also corroborated by dynamic light scattering (DLS) data.

The average radius of the Hb-PAA-1k obtained from the DLS measurements, indicated average diameters of 150, 145, and 115 nm, synthesized at pH 6, 7, and 8, respectively. Similarly, Hb-PAA-10k samples synthesized at pH 6, 7, and 8, indicated average diameters to be 160, 180, and 200 nm, respectively. These values closely match those obtained from the TEM micrographs above. Taken together, the TEM and DLS data show that polymer conjugation to the protein produced nanocapsules of Hb but not gels, microgels, or nanogels.

### Example 4

### Enzyme Structure and Activities

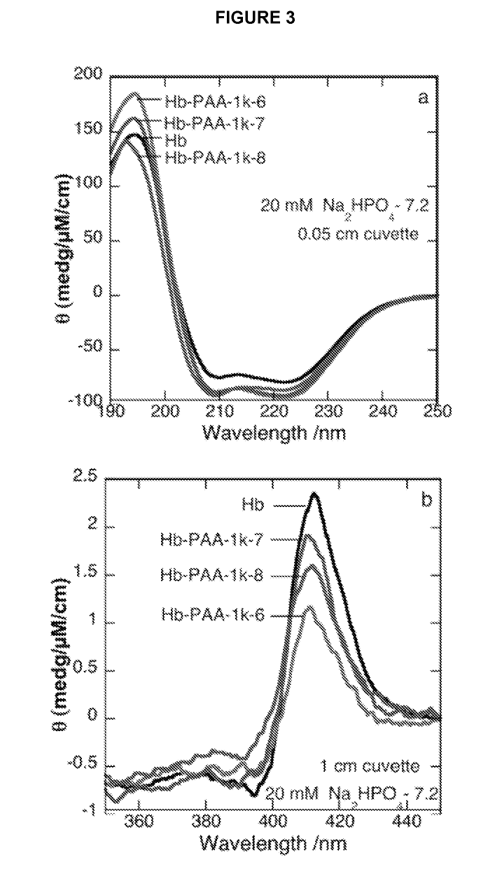

Native-like structure of the enzyme is critical for the retention of its biological activity, and we used circular dichroism (CD) spectroscopy to assess enzyme structure subsequent to encapsulation. The CD spectra of the Hb-PAA conjugates in the far-UV region (FIG. 3a) as well as the Soret region (FIG. 3b) are essentially the same as that of Hb, and these indicated significant retention of its native-like structure. Linking of nearly seven PAA chains per Hb molecule did not influence its structure to significant extent and this observation suggests the benign nature of PAA on Hb structure. Only minor changes are indicated, and these did not adversely influence peroxidase-like activity of Hb.

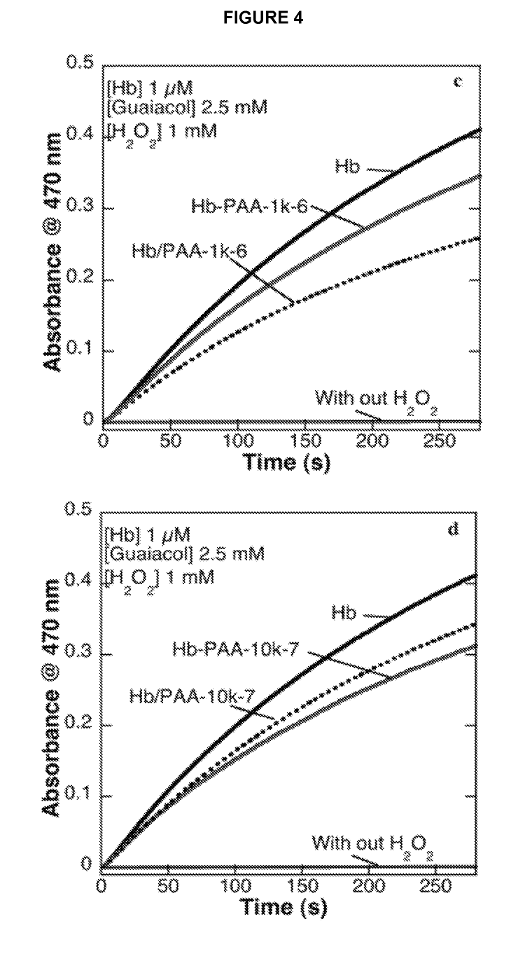

Hb activity was monitored by the addition of guaiacol (substrate) and hydrogen peroxide (oxidant), and formation of the colored product has been monitored by its absorption at 470 nm as a function of time (FIG. 4). Initial rates of the catalytic reaction and specific activity were obtained from these kinetic traces. For example, the initial rate of product formation catalyzed by Hb-PAA-1k (FIG. 4a) and Hb-PAA-10k (FIG. 4b) nanocapsules (green lines) are comparable to the corresponding physical mixtures of Hb and PAA (FIG. 4a, b, dotted lines) and they retained initial velocities of about 90% of untreated Hb (black curves). PAA did not catalyze the reaction (data not shown) and there has been no reaction in the absence of hydrogen peroxide (red curves). Thus, conjugation of PAA to Hb and the formation of the nanocapsules did not cause a significant decrease in the activity of Hb-PAA. Crosslinking of the polymer with the protein is expected to increase the rigidity of the latter and increased rigidity is expected to lower the activity for most proteins and enzymes. However, the activities of the nanocapsules did not decrease substantially, and thereby suggest that the increased rigidity does not adversely influence the activity or that the porous polymer film allows sufficient flexibility necessary for the catalysis of the oxidation reaction.

TABLE 1
Specific activities of Hb-PAA nanocapsules measured at
room temperature

| Sample | Specific activity | % Activity |
|---|---|---|
| Hb | 2.5 ± 0.3 | 100 |
| Hb/PAA-1k | 2.3 ± 0.07 | 92 |
| Hb-PAA-1k-6 | 2.4 ± 0.5 | 96 |
| Hb-PAA-1k-7 | 2.0 ± 0.2 | 80 |
| Hb-PAA-1k-8 | 1.4 ± 0.07 | 56 |
| Hb/PAA-10k | 2.0 ± 0.07 | 80 |
| Hb-PAA-10k-6 | 2.7 ± 0.4 | 108 |
| Hb-PAA-10k-7 | 2.6 ± 0.07 | 104 |
| Hb-PAA-10k-8 | 2.4 ± 0.1 | 96 |

Conjugating carbonic anhydrase (CA) to polyacrylic acid resulted in the corresponding CA-PAA nanocapsules, with their specific activity shown in Table 2. The specific activity is measured at room temperature. SS stands for steam sterilized samples (heating in an autoclave at 120° C., for 1 minute, 15 psi, indicated as x-ss). Other samples treated at 60 or 90° C. for 15 minutes and cooled to room temperature are indicated as x-60° C. or x-90° C., respectively, before measuring their activities. Activities are given as absorbance units per second per micromol of the protein.

TABLE 2
Specific activities of carbonic anhydrase (CA) conjugated to PAA

| | Specific activity |
|---|---|
| Sample | (AU/s/μM) |
| CA | 7.0 × 10⁻³ |
| CA-PAA-1k-6 | 2.0 × 10⁻³ |
| CA-PAA-1k-7 | 1.5 × 10⁻³ |
| CA-PAA-1k-8 | 1.2 × 10⁻³ |
| CA-PAA-10k-7 | 1.6 × 10⁻³ |
| A-ss | 1.5 × 10⁻⁴ |
| CA-PAA-10k-6-60° C. | 6.0 × 10⁻³ |
| CA-PAA-10k-8-60° C. | 1.4 × 10⁻³ |
| CA-PAA-10k-6-90° C. | 4.0 × 10⁻³ |
| CA-PAA-10k-8-90° C. | 4.0 × 10⁻³ |
| CA-PAA-1k-6-ss | 4.0 × 10⁻⁴ |
| CA-PAA-1k-8-ss | 1.3 × 10⁻⁴ |

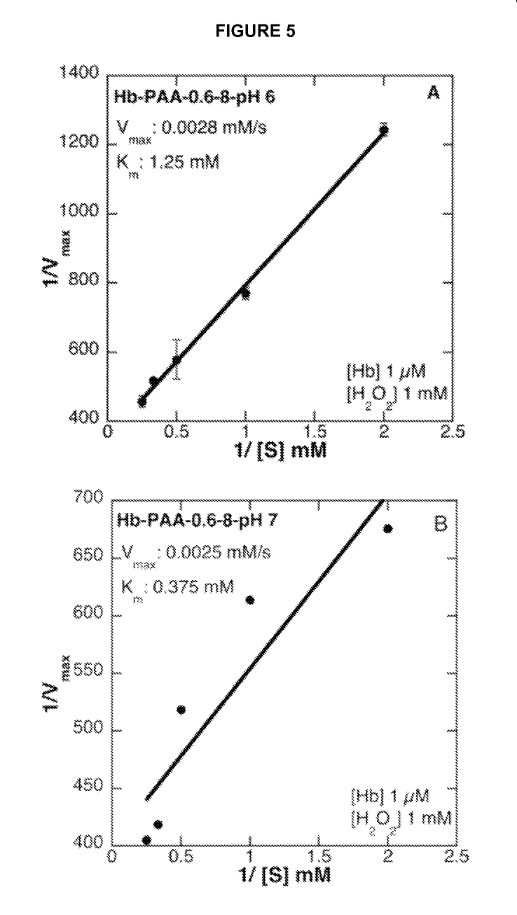

Initial reaction rates from the above data were used to construct Michaelis-Menton plots (FIG. 5) to extract the enzymatic parameters, Michaelis constant (Km) and the maximum velocity (Vₘₐₓ). Hb-PAA-1k-6 and Hb-PAA-1k-7 indicated Vₘₐₓ of 0.0028 mM/s and 0.0025 mM/s and Km of 1.25 mM and 0.375 mM, respectively, which are comparable to that found for unmodified Hb (Vₘₐₓ and Km of 0.0026 mM/s and 0.500 mM, respectively) under similar conditions of buffer, ionic strength, temperature and pH (Table 3). These outcomes are consistent with the CD data, and the retention of native-like structure, and retention of a high degree of activity is essential for biological applications of Hb-PAA nanocapsules.

TABLE 3
Michalis-Menten parameters for the Hb-PAA conjugates

| Sample | Km (mM) | Vₘₐₓ (mM/S) |
|---|---|---|
| Hb | 0.507 | 0.0021 |
| Hb-PAA-1k-6 | 1.25 | 0.0028 |
| Hb-PAA-1k-7 | 0.375 | 0.0025 |

### Example 5

### Thermal Stabilities

While the melt curves provide a quick measure of thermal stability, DSC provides a direct quantitative measure of the thermodynamic parameters and information regarding the reversibility of the denaturation. Nano II differential scanning calorimeter (DSC) 6100 from calorimetry Sciences Corporation (CSC, Utah) was used to perform thermal denaturation experiments. The amount of heat required to increase the temperature of a sample with respect to a reference (Cp) was monitored in a series of heating and cooling scans from 20 to 120° C. at a scan rate of 2° C./min. Thermodynamic parameters (ΔH, and Tₘ) were obtained from the DSC traces.

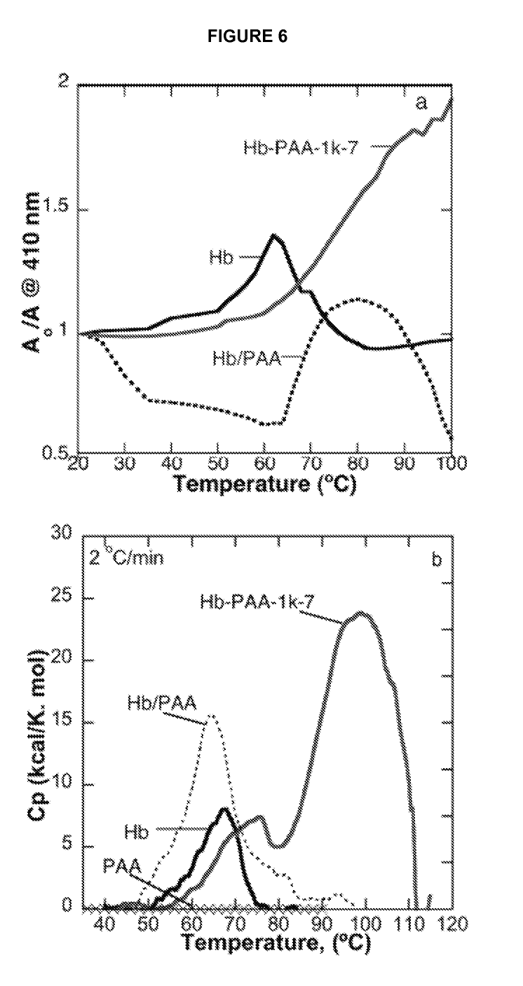

A plot of the Soret absorbance at 407 nm as a function of temperature, normalized with respect to initial absorbance of the sample (FIG. 6a), indicated a sharp change around 65° C. for Hb, and this is consistent with the denaturation temperature of Hb followed by its precipitation, under the same conditions of buffer, pH and ionic strength. On the other hand, the melt curve for Hb-PAA-1k-7 (FIG. 6a) indicated the beginning of a change around 60° C., which continued till 100° C. and did not reach a plateau even at this high temperature. The physical mixture of Hb and PAA indicated some stabilization followed by denaturation and precipitation. Thus, Hb-PAA-1k-7 indicated improved thermal stability when compared to that of Hb or Hb/PAA physical mixture. Thus, the nanocapsules protect the encapsulated protein to a significant extent and this served as a simple strategy to enhance thermal stability of Hb.

To quantify the thermodynamic parameters of protein denaturation, the DSC curves of Hb, Hb/PAA physical mixture and Hb-PAA conjugates (FIG. 6b) were recorded and the denaturation temperatures (Tₘ) have been determined. The DSC profile of Hb, for example, indicated Tₘ of 68° C., which is in agreement with literature value. While the Tₘ of Hb/PAA physical mixture is similar to that of Hb, the nanocapsules indicated much greater stability with no clear indication of complete denaturation. For example, denaturation of Hb-PAA-1k-7 began around 60° C., continued until 110° C., still not complete, and such high thermal stability is unusual for ordinary proteins.

The denaturation enthalpies (ΔH) were also estimated from the DSC curves, and these are shown in Table 4. ΔH is model independent, and its evaluation does not require that denaturation be reversible. The ΔH for the denaturation of the nanocapsules (130-540 kcal/mol) are significantly higher than those of Hb (130 kcal/mol), as well as Hb/PAA physical mixture (150 kcal/mol). The improved thermal stabilities of the nanocapsules clearly show that the polymer nanowalls protect the protein from thermal denaturation, and increase in thermal stabilities are likely due to the decrease in the conformational entropy of the encapsulated protein, as in the case of proteins dissolved in ionic liquids, while maintaining significant activity.

TABLE 4
Thermodynamic parameters for the denaturation of Hb nanocapsules

| | | | Hb-PAA- | Hb-PAA- | Hb-PAA- |
|---|---|---|---|---|---|
| | Hb | Hb/PAA | 1k-6 | 1k-7 | 1k-8 |
| Tₘ/° C. | 68 ± 0.2 | 68 ± 0.2 | 68 ± 0.2 | 68 ± 0.2 | 68 ± 0.2 |
| ΔH/kcal/mol | 140 ± 5 | 140 ± 5 | 140 ± 5 | 140 ± 5 | 140 ± 5 |

### Example 6

### Stability Towards Heat and Steam Sterilization

A JASCO model J710 spectropolarimeter was used to record the CD spectra. Scan rates were 50 nm/min with a step resolution of 1 nm/data point. Several scans (8-16) were averaged for each sample (0.05 and 1 cm path length) and data plotted using Kaleidagraph 3.0. Samples for CD were prepared as mentioned above and signals have been normalized with respect to concentrations of individual samples.

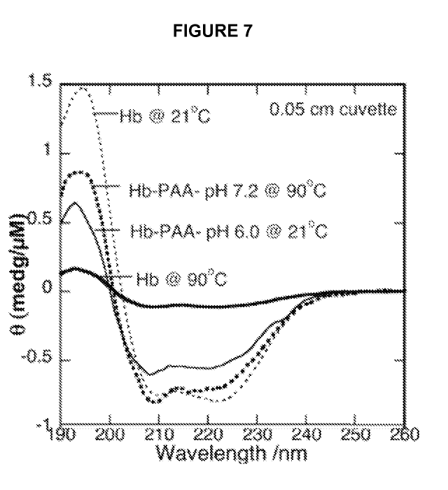

The CD spectra of the nanocapsules were examined, after they have been subjected to heat treatment at 90° C. for 15 minutes and then cooled back to room temperature. The heat-treated nanocapsules showed extensive retention of their UV CD spectra and hence, their secondary structure has been retained even after heating to 90° C. while heat treated Hb lost almost 80% of its CD signal (FIG. 7). These data indicate that the soft, hydrophilic polymer shell around the protein stabilizes it to a significant extent and the denatured protein refolds upon cooling to room temperature.

Hb activity was followed by literature method, with minor modifications. Product absorbance at 470 nm was monitored as a function time of after the addition of H₂O₂ (1 mM) to Hb (5 μM) and o-methoxyphenol (2.5 mM). Initial rates and Michaelis-Menten parameters were calculated from the kinetic data by following standard protocols. The activities of samples after steam sterilization at 120° C. for 1 min at 2 atm and cooling down to room were measured and analyzed in a similar way to untreated samples.

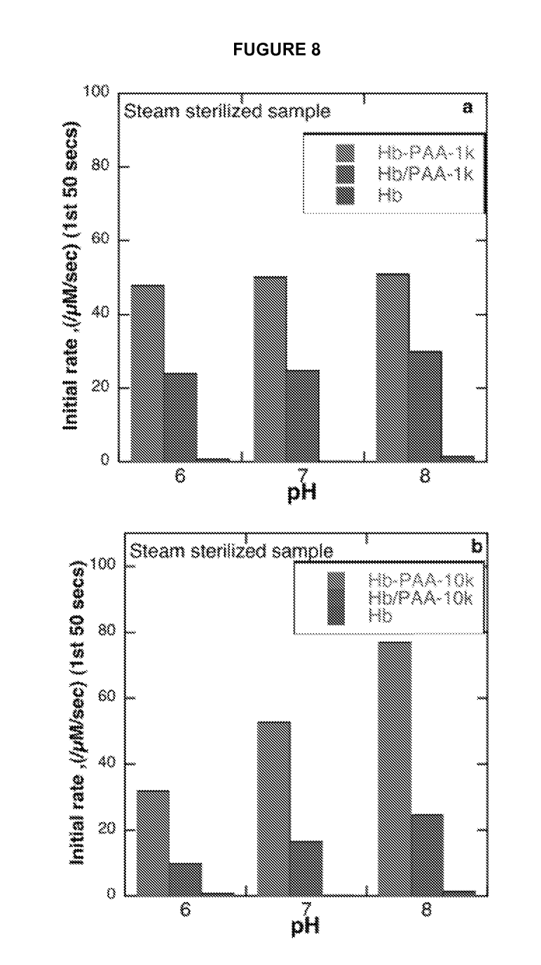

Specific activities of Hb, Hb/PAA and Hb-PAA-1k and Hb-PAA-10k samples, after steam sterilization (120° C., 1 min, 2 atm), are compared in FIG. 8. Steam sterilization resulted in nearly 90% loss of Hb activity. Similarly, the physical mixture of Hb/PAA also indicated a loss of 75% of its activity after steam sterilization while Hb-PAA nanocapsules retained a significant fraction of their original activities. For example, Hb-PAA-1k nanocapsules (FIG. 8a) retained as much as 84% of their initial activities, independent of the pH used for the synthesis, and Hb-PAA-10k (FIG. 8b) samples also retained a large fraction of their activities. However, the Hb-PAA-10k-8 sample retained much greater activities than all the others. Overall, nanocapsules are highly resistant to deactivation of steam sterilization conditions.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A nanocapsule comprising a protein moiety covalently attached to a polymer, wherein the polymer encapsulates the protein in a nanoshell.

2. A nanocapsule according to claim 1, wherein the nanocapsule is about 10 to about 200 nm in diameter.

3. A nanocapsule according to claim 2, wherein the nanocapsule is about 10 to about 100 nm in diameter.

4. A nanocapsule according to claim 1, wherein the nanoshell is a thin film of polymer that is about 1 to about 100 nm thick.

5. A nanocapsule according to claim 4, wherein the thin film of polymer is about 10 to about 50 nm thick.

6. A nanocapsule according to claim 1 wherein the protein moiety is covalently attached to a polymer at one or more sites on the protein and at one or more sites on the polymer.

7. A nanocapsule according to claim 6 wherein the attachment is of the protein to the polymer at one or more sites that are randomly located on the protein.

8. A nanocapsule according to claim 6 wherein the attachment of the protein to the polymer is through a lysine side chain of the protein moiety.

9. A nanocapsule according to claim 1, wherein the polymer is a polyacid.

10. A nanocapsule according to claim 9, wherein the polyacid comprises a polyacid selected from the group consisting of polyacrylic acid, polyacrylic acid sodium salt, poly(acrylic acid-co-maleic acid), poly(methyl vinyl ether-alt-maleic acid), poly(acrylamide-co-acrylic acid), poly(lactic acid), poly(glycolic acid), and combinations thereof.

11. A nanocapsule according to claim 10, wherein the polyacid comprises polyacrylic acid.

12. A nanocapsule according to claim 1, wherein the polymer has a molecular weight between about 500 and about 20,000.

13. A nanocapsule according to claim 12, wherein the polymer has a molecular weight between about 15,000 and about 20,000.

14. A nanocapsule according to claim 1, wherein the protein is an enzyme or a globin.

15. A nanocapsule according to claim 14, wherein the protein is met-hemoglobin, hemoglobin, glucose oxidase, carbonic anhydrase, or lipase.

16. A process for preparing a nanocapsule according to claim 1, comprising reacting a polymer with a protein in a spontaneous reaction.

17. A process for preparing a nanocapsule according to claim 1, comprising reacting a polymer with a protein in the presence of: a catalyst, a coupling reagent, light, higher-than-ambient temperature, another protein, an enzyme, or a combination thereof.

18. A process according to claim 17, wherein the coupling reagent is a carbodiimide selected from: N,N′-dicyclohexylcarbodiimide, N,N′-diisopropylcarbodiimide, and 1-ethyl-3-(3-dimethylaminopropyl)carbodiimide.
